(19) United States
(12) Patent Application Publication
(10) Pub. No.: US 2006/0259053 A1
(43) Pub. Date: Nov. 16, 2006

(54) Automated ophthalmic device for performance of capsulorhexis

(76) Inventor: Jeylan A. El-Mansoury, Penn Valley, PA (US)

Correspondence Address: John S. Munday, Esquire, PO Box 423, Isanti, MN, 55040, US

(21) Appl. No.: 11/395,083

(22) Filed: Mar. 31, 2006

Related U.S. Application Data
(63) Continuation-in-part of application No. 11/102,155, filed on May 16, 2005.
(60) Provisional application No. 60/560,913, filed on Apr. 9, 2004.

(51) Int. Cl. A61F 9/00

(52) U.S. Cl. 606/166

(57) ABSTRACT

A mechanical surgical device and device are provided for allowing the user to form a circular incision in an intraocular tissue, such as the anterior capsule of the eye, as part of an anterior capsulorhexis. Unlike the prior art, the device of the present invention is comprised of mechanical components, where the mechanical components drive a cutting member in a motion around a fixed pivot point to perform a continuous curvilinear capsulorhexis. Where the pivot point is the location of the slit incision in the cornea or the sclera through which the cutting tip is inserted to access the anterior capsule bag. The present device is hand held, compact and relatively light in weight. The present capsulorhexis device requires only a 1 mm incision in the corneal or scleral tissue. The present invention does not require the manual skill of the user to perform a circular incision.

### FIELD OF THE INVENTION

This invention relates to a mechanical surgical device and method for the purpose of forming a curvilinear incision in tissue. More particularly, this invention relates to a device and method which are used in ophthalmic surgery for accomplishing capsulorhexis in the anterior capsule of the eye. This is a continuation-in-part of my co-pending application filed Apr. 8, 2005, having Ser. No. 11/102,155, the disclosure of which is incorporated herein by reference.

### BACKGROUND OF THE INVENTION

Cataract, a common eye disease is the opacification of the material within the intraocular lens of the eye. Eye surgeons and others have developed techniques and instruments to extract the cataract and replace the natural opacified intraocular lens with an artificial intraocular lens, this procedure is called cataract surgery. Cataract surgery is a multi-step surgical procedure, with the main steps being capsulotomy or capsulorhexis, phacoemulsification and intraocular lens replacement.

Capsulorhexis or capsulotomy is a technique that involves making a slit incision (generally around 3 mm) in the cornea of the eye, through which an instrument is entered and the same or alternate instrument is used to form a 6 mm circular incision in the anterior capsule bag.

Capsulorhexis, also known as continuous curvilinear capsulorhexis, is a technique that defines a circular incision that is formed through continuous tearing of the anterior capsule bag, whereas capsulotomy defines a circular incision that is formed by non-continuous slicing through the anterior capsule bag.

Capsulorhexis utilizes forceps to tear a flap in the anterior capsule bag, grasp the flap and manually rotate it to form the circular incision. Opening and closing the forceps during this technique may cause trauma to the cornea. Trauma to the eye may also be caused by pressure exerted on the eye by the surgeon during the procedure.

Additional complications may be caused by a non-circular tear of the anterior capsule bag. Difficulties with this procedure include size and shape control of the incision; it is difficult to manually adhere to the 6 mm circular requirement.

Capsulotomy utilizes a needle cystotome to slice through the anterior capsule bag forming rough and jagged edges as it slices, just as a can opener would when opening a tin can. Rough and jagged edges may cause complications in the surgical procedure. The present invention provides a surgical device which is particularly useful in performing the capsulorhexis.

Prior art has presented instruments which have tried to improve both capsulotomy and capsulorhexis procedures, but have still relied on the manual skill of the surgeon to perform a smooth curvilinear 6 mm incision. U.S. Pat. No. 6,306,155 describes a forceps having a replaceable hub allowing a constant grip of the cornea and utilizing the forceps as both a forceps and a cystotome. U.S. Pat. No. 5,167,618 describes forceps which may also function as a cystotome. U.S. Pat. No. 4,708,138 describes a surgical cutting knife with a rotating blade.

Other prior art has presented instruments which have tried to improve both capsulotomy and capulorhexis procedures, which do not rely on the manual skill of the surgeon to perform a smooth curvilinear 6 mm incision, but rather provide an automated instrument that is driven by an ultrasonic, hydraulic, vacuum, electromagnetic waves or other power source. These instruments are complex and provide unwanted energy and forces to the eye.

U.S. Pat. Nos. 6,165,190, 5,860,994 and 5,261,923 describe an automated system comprising a cutting blade driven by a motor or motor-like device to perform the incision. These systems are complex requiring the device be attached to a power supply.

U.S. Pat. No. 5,296,787 describes an apparatus in which the cutting member works in conjunction with an ultrasonic power source to perform the curvilinear incision. This system is not only complex but may provide unwanted energy to the eye. Additionally this system requires that the cutting member be bent in order to fit through the corneal slit incision, which further complicates the system.

U.S. Pat. No. 5,569,280 describes an ophthalmic template that requires attachment to a vacuum source to perform the opening in the anterior capsule. Additionally this system requires that the cutting member be bent in order to fit through the corneal slit incision, which further complicates the system.

U.S. Pat. No. 5,873,883 describes a surgical apparatus that requires attachment to a fluid source and uses incompressible fluid to incise a smooth, continuous curvilinear aperture in the anterior capsule of the eye. The cutting member of the apparatus flexes and deforms as it is inserted into the incision, further complicating the apparatus.

U.S. Pat. No. 5,346,491 describes an electrical capsulotomy device for eye microsurgery. This device requires a pulsed high frequency current to be fed to the instrument. This system is not only complex but may provide unwanted energy to the eye.

U.S. Pat. Nos. 4,367,744 and 4,481,948 describe cauterizing rings driven by an electrical power source. This system is not only complex but may provide unwanted energy to the eye.

It would be of great advantage in the art if a device and method could be developed that uses a mechanical component to drive the cutting member.

It is therefore an advantage that the device of the present invention does not require the cutting tip of the instrument to be deformed in order to pass through the sclero-corneal or corneal incision.

Another advantage that the device of the present invention requires only a 1 mm sclero-corneal or corneal incision to be made. The incision is relatively small compared to that required by prior art, about 3 mm. This provides faster recovery of the eye and reduction or elimination of stitches.

Yet another advantage of the present invention is that the circular incision is formed without jagged edges or tears. The advantage includes but is not limited to reduction or elimination of complications in the cataract surgical procedure.

Other advantages will appear hereinafter.

### SUMMARY OF THE INVENTION

It has now been discovered that the above and other objects of the present invention may be accomplished in the following manner. The unique aspect of this invention is the use of a novel automated ophthalmic device for performing a capsulorhexis which foregoes the disadvantages associated with prior art. Unlike the prior art, the device of the present invention is comprised of mechanical components, where the mechanical components drive a cutting member in a motion around a pivot point to perform a continuous curvilinear capsulorhexis. Where the pivot point is the location of the corneal, scleral or sclero-corneal incision, through which cutting tip is inserted to access the anterior capsule bag.

In the present invention, the mechanical components are fully contained internally within the device without the requirement of external attachments such as power, hydraulic, vacuum, electromagnetic waves or ultrasonic sources. The present invention has a smaller overall size, being lighter in weight and being more cost effective. The cutting member of the device of the present invention may be replaceable, to permit replacement of a damaged or used tip without replacement of the entire device. The device maintains a pivot point without the traditional ball-and-socket pivot, so that there is effectively the elimination of interference with the eye during capsulorhexis.

In the present invention, the user activates or loads the device with mechanical energy and receives simple feedback from the device to know when activation, that is loading, is complete. This empowers the user with the knowledge that the device is in its active form and caution should now be taken to avoid accidental triggering.

Manually triggering releases the stored mechanical energy from the device and is accomplished by sliding or pushing an ergonomically located trigger button. Thus the user knows that the incision has been performed. The manual skill of the surgeon is not required to perform a curvilinear incision. The device eliminates performing a non-circular incision or tear in the capsular bag. Non-circular incisions or tears result in complications in the cataract surgical procedure.

### BRIEF DESCRIPTION OF THE DRAWINGS

For a more complete understanding of the invention, reference is hereby made to the drawings, in which:

FIG. 1 is a side view of the automated capsulorhexis device of the present invention

FIG. 2 is a top view of the automated capsulorhexis device of the present invention

FIG. 3 is a view of FIG. 1, rotated 60 degrees along the horizontal, 12 degrees along the vertical and 35 degrees along the normal

FIG. 4 is an enlarged view of the cutting member of FIG. 3

FIG. 5 is the image of FIG. 1 including site alignment

FIG. 6 is the image of FIG. 2 including site alignment

FIG. 7 is the image of FIG. 3 including site alignment

FIG. 8 is an enlarged view of the assembly of FIG. 3, without the presence of the housing case and rear housing cover, and including only a partial view of the handle

FIG. 9 is an enlarged view of FIG. 1, rotated 60 degrees along the vertical and 30 degrees along the normal, illustrating the components related to depth control

FIG. 10 is an enlarged view of FIG. 1 illustrating the components related to the ‘off-set’ pivot point of the device

FIG. 11 is an enlarged view of FIG. 1, rotated 90 degrees along the vertical and 30 degrees along the horizontal, illustrating some the components of the device related to activation of the device

FIG. 12 is an enlarged view of FIG. 5 illustrating the components of the device related to triggering of the device

FIG. 13 is a top view of an illustration of the motion around a pivot point followed by the cutting member for performance of a continuous curvilinear capsulorhexis

FIG. 14 is an enlarged view of FIG. 9 illustrating the components related to guiding the motion of a cutting member during release of mechanical energy and halting the release of the mechanical energy

### DETAILED DESCRIPTION OF THE PREFERRED EMBODIMENT

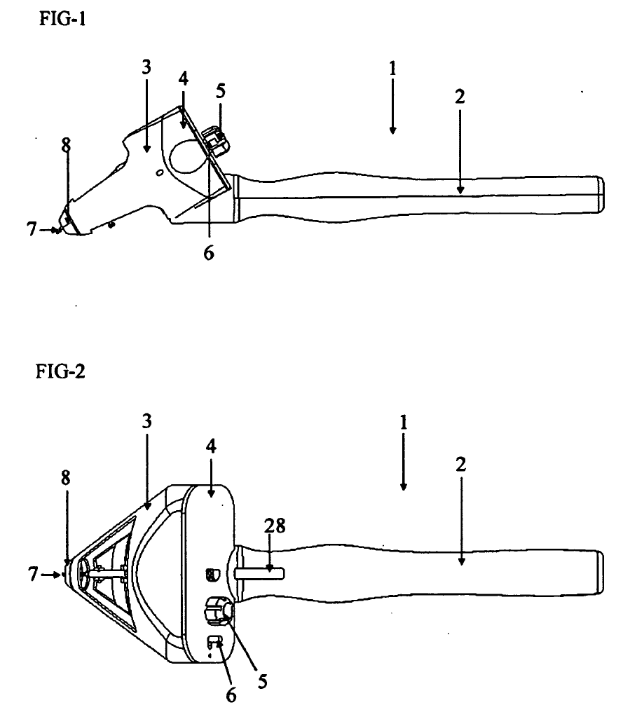

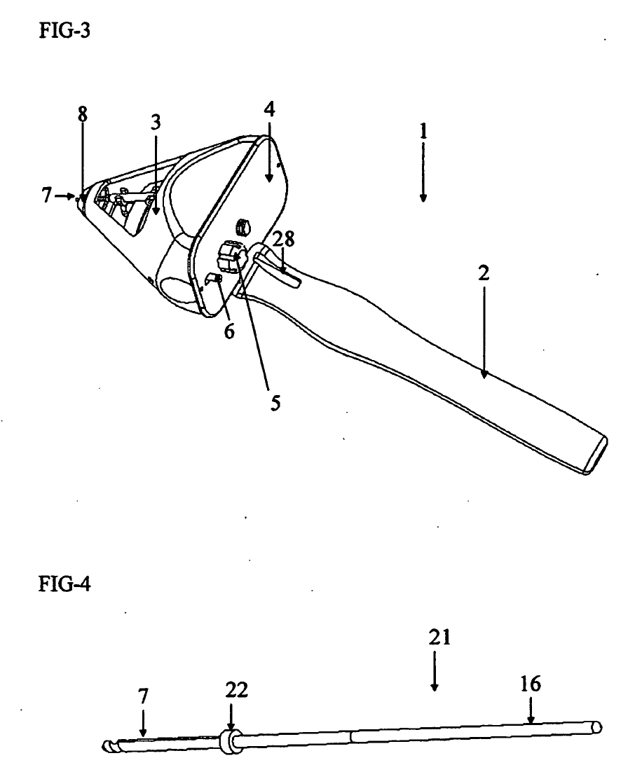

Referring now to the Drawings and in particular to FIG. 1, FIG. 2 and FIG. 3, there is shown an automated capsulorhexis device 1, having a handle 2, a housing case 3, a rear housing cover 4, a setting dial 5, a lock button 6, a user level 28, a guide front 8 and a cutting tip 7. Cutting tip 7 is the proximal end of cutting member 21.

Cutting member 21 is shown in FIG. 4. Cutting member 21 is a single piece component having cutting tip 7 at the proximal end, end rod 16 at the distal end and cutting member collar 22 between cutting tip 7 and end rod 16. Cutting tip 7 may have one of several designs. Alternatively, cutting member 21 and cutting tip 7 may be comprised of two or more components

Handle 2 is intended to be ergonomically designed for a comfortable grasp of the device by the user. Handle 2 may have one of several designs. Handle 2 comprises user level 28. User level 28 indicates orientation of the device. It is an advantage of the present invention that the user be knowledgeable that the device is properly oriented for use.

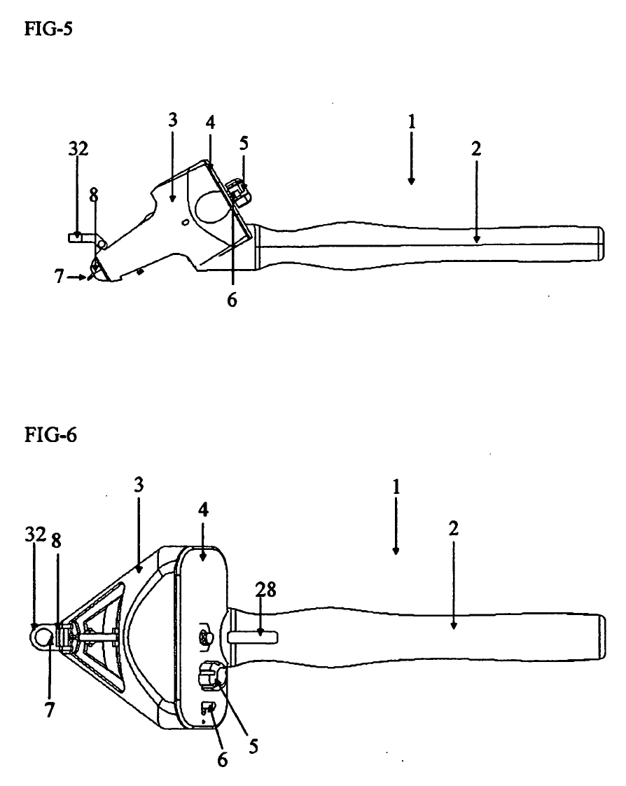

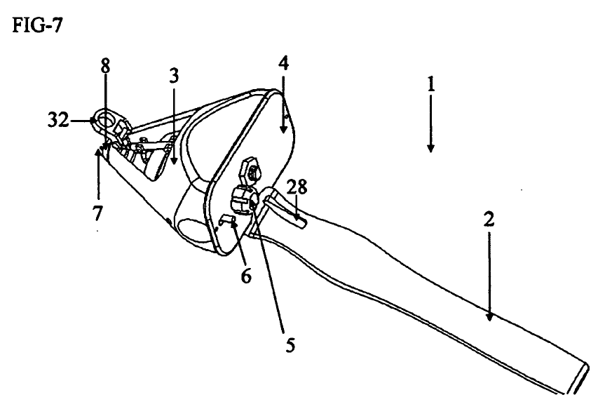

The device of the present invention may or may not comprise site alignment 32. Site alignment 32 may be seen in FIGS. 5, 6, and 7. Site alignment 32 indicates orientation of cutting tip 7, such that the circular incision is centered or properly positioned with respect to the surface of the anterior capsule bag. In an alternate ergonomic design, site alignment 32 may comprise user level 28.

The device of the present invention forms a circular incision, otherwise known as a continuous curvilinear capsulorhexis, in the anterior capsule of the eye. Where the circular incision is without jagged edges or tears and where the precision of the circular 1 incision does not require the manual skill of the user and where only a 1 mm slit incision is required in the cornea or sclera. It is an advantage of the present invention that the continuous curvilinear capsulorexhis is formed without jagged edges or tears. The advantage includes but is not limited to reduction or elimination of complications in the cataract surgical procedure. It is an advantage of the present invention that the corneal, scleral or sclero-corneal incision is relatively small, 1 mm, compared to that required by prior art, about 3 mm. The advantage includes but is not limited to faster recovery of the eye and reduction or elimination of stitches. It is an advantage of the present invention that the circular incision does not require the manual skill of the user. The advantage includes but is not limited to elimination of performing an inaccurate circular incision, where the incision may be non-circular or may be of an incorrect size.

The device of the present invention is comprised of mechanical components, where the mechanical components drive cutting member 21 in a motion around a fixed pivot point to perform a continuous curvilinear capsulorhexis.

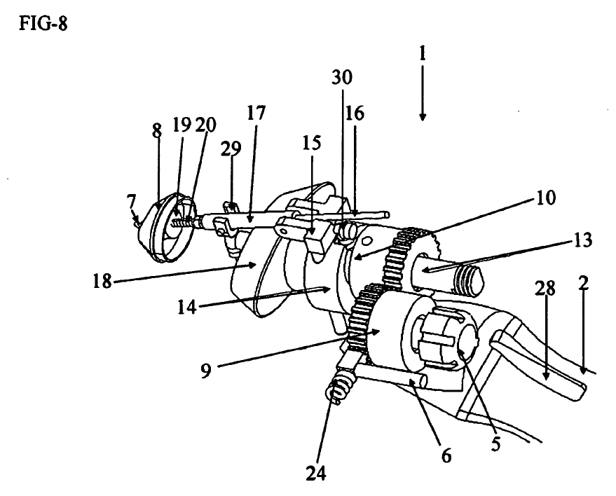

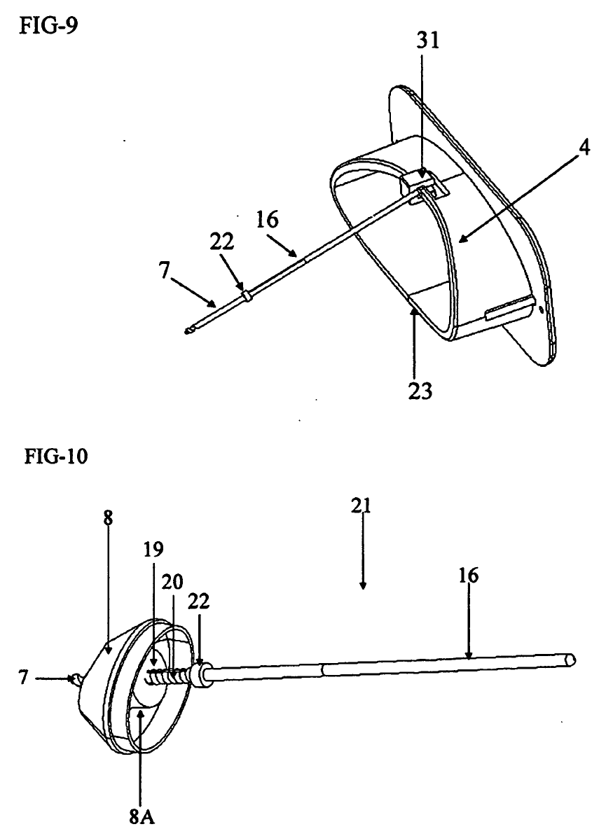

The assembly of the mechanical components of the device of the present invention is shown in FIG. 8; partial assemblies of the mechanical components are better illustrated in FIGS. 9, 10, 11 or 12. It will be apparent by those skilled in the art that modifications, additions to, replacements and rearrangements of the mechanical components are possible without diverting from the scope of the invention. The mechanical components include setting dial 5, which interfaces with input gear 9, which in turn interfaces with output gear 10, output gear 10 interfaces with elliptical arm 14, which in turn is attached to coil holder 11 which in turn holds the end of coil spring 12, coil spring 12 wraps around keyed protrusion 13, keyed protrusion 13 is held by elliptical arm 14, circular translator 15 is held within elliptical arm 14, circular translator 15 is attached to drive shaft 17, proximal shaft holder 29 is a front compression spring which holds drive shaft 17, distal shaft holder 30 is a rear extension spring which also holds drive shaft 17, drive shaft 17 holds cutting member 21, the distal end of cutting member 21 is end rod 16, end rod 16 interfaces with depth control path 23, depth control path 23 is an engraved feature in rear housing cover 4, elliptical arm 14 interfaces with elliptical CAM 18, the proximal end of cutting member 21 is cutting tip 7, cutting tip 7 interfaces with guide pivot 19, pivot spring 20 wraps partially around cutting tip 7, pivot spring 20 interfaces with cutting member collar 22 and with guide pivot 19 and guide pivot 19 interfaces with guide front 8.

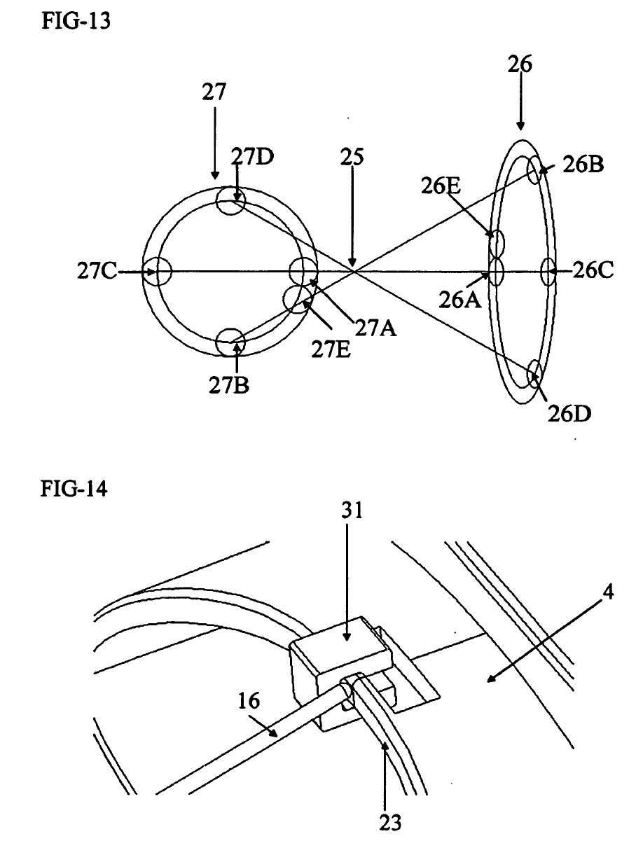

The device of the present invention requires a corneal, scleral or sclero-corneal incision of only 1 mm to be made in the eye. The only component of the apparatus that is inserted through the incision is cutting tip 7. Cutting tip 7 is manually inserted through the 1 mm incision at an angle with respect to the horizontally oriented anterior capsule surface. Cutting tip 7 is manually positioned through the surface of the anterior capsule bag, where the circular incision is to start. Cutting tip 7 may be marked to indicate to the user appropriate depth position of cutting tip 7 through the anterior capsule bag. It is preferred that the circular incision is to start at the point closest to the 1 mm incision (start position 27A is shown in FIG. 13 and is described below). Depth control path 23 will also account for changes from an ellipse in a non-horizontal plane to a circle in the horizontal plane assuring that cutting tip 7 is always tangential to the circular incision; this concept will be further explained below.

To maintain the corneal, scleral or sclero-corneal incision at a minimum size while performing the continuous curvilinear capsulorhexis, cutting member 21 is rotated around a pivot point. Where the pivot point is the location of the corneal, scleral or sclero-corneal incision through which cutting tip 7 is inserted to access the anterior capsule bag.

To prevent jagged edges or tears in the surface of the anterior capsule wall while performing a continuous curvilinear capsulorhexis, the device of the present invention requires that cutting tip 7 maintain constant contact with the anterior capsule wall. For cutting tip 7 to maintain constant contact with the anterior capsule wall, cutting tip 7 must move in a circular motion along a horizontal plane.

The motion of cutting member 21 around a pivot point and the motion of cutting tip 7 in a circular motion along a horizontal plane are described with reference to FIG. 13. Ideally, whatever direction of motion distal end 16 follows should translate to and allow cutting tip 7 to be set in a circular motion along the horizontal plane. FIG. 13 illustrates the motion of cutting member 21 around a pivot point translating an elliptical motion along a non-horizontal plane 26 into a circular motion along a horizontal plane 27. An elliptical motion 26 in a non-horizontal plane when rotated around pivot point 25 will not translate into a circular motion along a horizontal plane 27 unless cutting member 21 travels further horizontally at some points in comparison to other points. FIG. 13 illustrates the relationship of various locations, 26A, 26B, 26C, 26D, and 26E, along the circumference of the ellipse in a non-horizontal plane to various locations, 27A, 27B, 27C, 27D and 27E, along the circumference of the circle in a horizontal plane. Point 27A represents the start point, and the location in the anterior capsule wall through which cutting tip 7 is inserted. Point 27B shows the position of cutting tip 7 if it were to travel one forth the distance required to perform a circular incision. Point 27C shows the position of cutting tip 7 if it were to travel one half the distance required to perform a circular incision. Point 27D shows the position of cutting tip 7 if it were to travel three forth the distance required to perform a circular incision. Point 27E shows the position of cutting tip 7 if it were to travel the entire distance required to perform a circular incision. Cutting tip 7 travels past start point 27A to end point 27E to ensure that a full circular incision has been made. Points 26A, 26B, 26C, 26D and 26E represent the location of end rod 16 along a non-horizontal plane with respect to the various locations, 27A, 27B, 27C, 27D and 27E, traveled by cutting tip 7 while performing the circular incision. FIG. 13 also illustrates the relative distance from one point to the next with respect to the pivot point, indicating that point 27C is further from pivot point 25 than either 27B or 27D and point 27A is the closest to pivot point 25.

For cutting tip 7 to move along the circumference of the circle on a horizontal plane and account for the relative changes in distance from pivot point 25, cutting member 21 must follow depth control path 23, as shown in FIG. 9. Rear housing cover 4 includes depth control path 23, wherein depth control path 23 is engraved in rear housing cover 4. End rod 16 of cutting member 21 interfaces with depth control path 23. Depth control path 23 functions as a guide for cutting member 21 and allows it to move the distance required for cutting tip 7 to move along a horizontal plane.

The device of the present invention is comprised of a stop system to allow cutting tip 7 to stop moving once it has reached end point 27E. The stop system is illustrated in FIG. 14. End rod 16 will follow depth control path 23 until it reaches stop control 31. End rod 16 will push stop control 31 into a pocket engraved in rear housing cover 4. End rod 16 will continue to push stop control 31 until stop control 31 has reached the end of the engraved pocket, at which point stop control 31 will stop moving and thus cause end rod 16 to stop moving, at end point 27E.

To additionally prevent jagged edges or tears the device of the present invention requires that the sharp edge of cutting tip 7 maintain constant contact with the anterior capsule wall for the entire duration while performing the circular incision. For the sharp edge of cutting tip 7 to maintain constant contact with the anterior capsule wall, cutting member 21 may be required to rotate one revolution with respect to itself as it travels the full circumference of the circular incision. The design of cutting tip 7 will determine the requirement for cutting member 21 to rotate with respect to itself. An explanation of the mechanics required for cutting member 21 to rotate with respect to itself is provided later.

The device of the present invention may maintain an “off-set” pivot point for cutting member 21. “Off-set” is a term used to explain a pivot point that is not constrained around a point as in a ball and socket pivot. FIG. 10 provides an illustration of the components of the device related to the “off-set” pivot point. Guide front 8 has a dome shaped interior 8A. Guide pivot 19 interfaces with dome shaped interior 8A of front guide 8. Dome shaped interior 8A and guide pivot 19 are concentric, their concentricity is what locates the “off-set” pivot point of the device. Pivot spring 20 assures that guide pivot 19 maintains a constant interface with dome shaped interior 8A of front guide 8. Pivot spring 20 wraps around a portion of cutting member 21 and is held in place by cutting member collar 22. Guide front 8 may have a snap fit, slip fit, screw fit or luer lock Interface with housing case 3. Although the device of the present invention may maintain a pivot point with the traditional ball and socket pivot, it is an advantage that the device may alternately maintain an “off-set” pivot point. The advantage includes but is not limited to elimination of device interference with the eye during capsulorhexis.

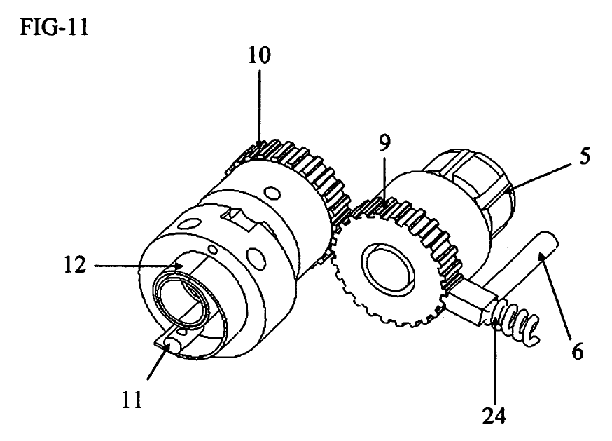

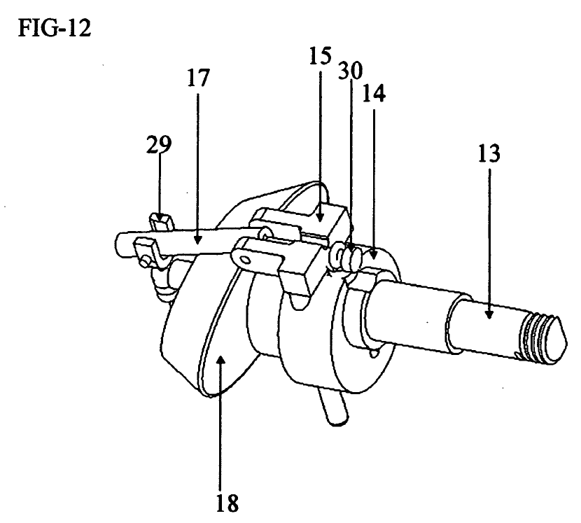

The present invention requires that the user activate the device prior to insertion and positioning of cutting tip 7 inside the eye. It is preferred that the user activate the device immediately prior to performing the continuous curvilinear capsulorhexis. FIG. 11 provides an illustration of the majority of the components related to activation of the device, reference to FIG. 12 may be required for illustration of the components not represented in FIG. 11. To activate the device, setting dial 5 is manually rotated by the user. Once the device is fully activated, setting dial 5 will no longer rotate, providing the user feedback that the device is now activated. Feedback is provided by a stop system encompassed within the device. Rotating setting dial 5 will simultaneously cause input gear 9 to rotate. Lock button spring 24 keeps lock button 6 pressed firmly against input gear 9, preventing input gear 9 from freely rotating back to its original position. Rotation of input gear 9 will simultaneously cause output gear 10 to rotate, which will simultaneously cause elliptical arm 14 to rotate, which will cause coil holder 11 to rotate, which will simultaneously wind coil spring 12 around keyed protrusion 13 and activate the apparatus. It is an advantage of the present invention that the user manually activates the device and is confident that the function is complete. The advantage includes but is not limited to empowering the user with the knowledge that the device is in its active form and caution should now be taken to avoid accidental triggering.

The device of the present invention allows the user to manually trigger the mechanical function of an activated device. The mechanical function allows cutting tip 7 to form a circular incision in the anterior capsule bag. FIGS. 11 and 12 provide an illustration of the majority of the components related to the mechanical function of the device. It is preferred that the user trigger the mechanical function of the activated device after insertion and positioning of cutting tip 7 inside the eye. To manually trigger the mechanical function of the activated device, the user shall disengage lock button 6 from input gear 9 by manually sliding lock button 6 to the side. Sliding lock button 6 to the side prevents lock button spring 24 from keeping lock button 6 pressed firmly against input gear 9 thus allowing input gear 9 to freely rotate to its initial inactive position as coil spring 12 is unwound. Unwinding of coil spring 12 will cause output gear 10 to rotate, which will cause elliptical arm 14 to rotate. As elliptical arm 14 rotates it will cause circular translator 15 to rotate. As circular translator 15 rotates it will move in and out of elliptical arm 14 allowing drive shaft 17, and thus cutting member 21, to turn around elliptical CAM 18. Drive shaft 17 is keyed and thus turns one revolution around itself, and thus cutting member 21 turns one revolution around itself, as drive shaft 17 completes one turn around elliptical CAM 18. Proximal shaft holder 29 and distal shaft holder 30 pull a normal force on drive shaft 17, allowing it to run smoothing on the surface of elliptical CAM 18. As cutting member 21 follows elliptical CAM 18, end rod 16 is set in a non-horizontal elliptical motion. The non-horizontal elliptical motion is translated into a horizontal circular motion at cutting tip 7. Cutting member 21 maintains an “off-set” pivot point as it rotates, and performs a circular incision in the anterior capsule wall. It is an advantage of the present invention that the user manually triggers the mechanical function of an activated device. The advantage includes but is not limited to empowering the user with the knowledge that the circular incision will be performed. It is an advantage of the present invention that the trigger is a lock button 6 ergonomically located on the device. The advantage includes but is not limited to convenient location for the user. It is an advantage of the present invention that the cutting function is mechanically controlled by mechanical components that are located internally within the device and that the device does not require external attachments. Where external attachments include but are not limited to power, hydraulic or ultrasonic sources. The advantage includes but is not limited to the device of the present invention having a smaller overall size, being lighter in weight and being more cost effective than devices of prior art.

The device of the present invention may be disposable or non-disposable. In the event that the device is non-disposable, cutting member 21 may be disposable or non-disposable. In the event that cutting member 21 is disposable it may be replaceable. FIGS. 9 and 10 illustrate majority of the device components related to replacing cutting member 21. Cutting member collar 22 is held within the device by a set screw. To replace cutting member 21 the user must unscrew the set screw that holds cutting member collar 22 and slide cutting member 21 out of drive shaft 17. In removing cutting member 21, guide pivot 19 and pivot spring 20 will also be removed. The user is to then slide a different cutting member 21 into drive shaft 17. Cutting member collar 22 will stop cutting member 21 from further advancing within drive shaft 17 once it is properly positioned. Once cutting member 21 is positioned within drive shaft 17, pivot spring 20 then guide pivot 19 are replaced. It is an advantage of the present invention that the cutting member be replaceable. The advantage includes but is not limited to replacement of a damaged or used tip without replacement of the entire device, with a result in cost savings.

It is an additional advantage of the present invention that the device components may be modified, replaced, rearranged or added to without diverting from the scope of the invention. The advantage includes but is not limited to improving performance, improving ergonomics, reducing friction, reducing wear and tear and controlling speed of the cutter.

Examples of modification, replacement, rearrangement and addition to the device components are briefly explained below.

Bearings may be added to the assembly of the device of the present invention. Herein bearing include but are not limited to, linear, radial, solid, ball and or any combination thereof. Addition of washers simultaneously with the bearings may be required. Addition of bearings may require modification of device components. Bearings may be added in various locations. Addition of bearings reduces frictional forces between device components which in turn decreases the wear and tear of device components and increases the life of the device.

Screws may be added for a tighter fix. Addition of screws may require modification of device components. Screws may be added in various locations, including but not limited to, at the location of proximal shaft holder 29 to allow for a tighter grip of drive shaft 17.

Device components may be rearranged to change the ergonomics of the device. Rearrangement of device components may require modification of other device components. Rearrangement of device components, includes but not limited to, shifting lock button 6 and lock button spring 24 such that they to sit in handle 2, shifting user level 28 from handle 2 such that it is combined with site alignment 32. Ergonomical changes to the device may make the device more user friendly.

Size of device components may be altered. Altering the size of device components may require modification of other device components. Device components that may be altered include but are not limited to, increase or decrease of the size of input gear 9 and output gear 10, which in turn may control the rotating speed of drive shaft 17 and other components (this concept is not illustrated in the figures attached). The size of device components may be altered to increase or decrease in the over all size of the device.

Springs may be replaced with springs of different properties. Replacement of springs with springs of different properties may require modification of other device components. Replacement of springs includes but is not limited to, coil spring 12, pivot spring 20 and lock button spring 24. Replacement of springs may lead to several changes, including but not limited to, altering the rotating speed of drive shaft 17 and other components (this concept is not illustrated in the figures attached).

Design of cutting tip 7 may be altered. Altering the design of cutting tip 7 may require modification of other device components. Design of cutting tip 7 may include but is not limited to spiral, triangular and flat, square and flat or rectangular and flat.

Design of cutting member 21 may be altered to accommodate a different mode of cutting tip. This may Include but Is not limited to a hollow tube that houses a laser source or some other energy or power emitting source that may be used to cut the intraocular tissue. The energy or power emitting source would be guided to cut the 6 mm incision by the assembly of the mechanical components of the devices, in a similar manner that it drives cutting tip 7. Modifications of the assembly of the mechanical components may be required to accommodate specific requirements of the energy or power emitting source. One preferred energy source is a laser, which is used in many medical applications. A preferred laser is the CO₂ laser.

Additional modification, replacement, rearrangement and addition to the device components may be required for alternate reasons, including but not limited to performing circular incisions of different sizes. The continuous curvilinear incision may be in the range of about 1.0 nm (nanometer) to about 10.0 mm (millimeter).

While particular embodiments of the present invention have been illustrated and described, it is not intended to limit the invention to any specific embodiment. The description of the invention is not intended to limit the invention, except as defined by the following claims.

1. A mechanical surgical device for performing a continuous curvilinear incision, comprising:
a cutting member having a cutting tip having means for mounting an energy source for cutting intraocular tissue, said tip being adapted to rotate;
an energy source mounted in said cutting member and adapted to cut intraocular tissue;
rotation means for rotating said cutting tip around a pivot point, said means including input means for rotating said tip about an axis in a first direction of rotation against a biasing means adapted to store said rotation; and
release means for releasing said biasing means to rotate said cutting tip about said axis in the opposite direction of rotation after said tip has engaged the surface on which the continuous curvilinear incision is to be made.

2. The device of claim 1, wherein said rotation means includes a setting dial and said release means includes a trigger button.

3. The device of claim 2, which further includes a stop means for limiting the rotation of said cutting means upon release thereon.

4. The device of claim 1, wherein said corneal, sclearal, or sclero-corneal incision is less than 1.1 mm.

5. The device of claim 1, wherein said continuous curvilinear incision may be in the range of about 1.0 nm to about 10.0 mm.

6. The device of claim 1, wherein said rotation means includes guide means for moving said cutting tip in a circular motion along a horizontal plan.

7. The device of claim 1, wherein said rotation means is housed in a housing case with a rear housing cover means for mounting one end of said cutting member.

8. The device of claim 1, which further includes handle means for mounting said rotation means, said handle means being formed to allow a user to precisely position for performing said incision.

9. The device of claim 1, wherein said energy source is a laser.

10. The device of claim 9, wherein said laser is selected from a continuous power source and a pulsed power source in the frequency range from ultraviolet to infrared.

11. The device of claim 1, wherein said cutting member is selected from a hollow tube, a fiber optic and a fiber optic within a hollow tube.

12. A mechanical surgical device for performing a continuous curvilinear incision, comprising:
a handle for use by a user;
an input gear mounted on said housing and having an input shaft for rotating said input gear;
an output gear engaged with said input gear and having an output shaft for being rotated by said output gear;
a spring mounted on said output shaft for storing rotation of said output gear by said input gear;
an elliptical arm attached to said spring and adapted to rotate to define an elliptical path when said spring is moved by said output shaft;
a drive shaft engaged with said elliptical arm and adapted to rotate to define a circular path when said elliptical arm moves in said elliptical path;
a cutting member on said drive shaft, said cutting member having a having a proximal end and a distal end, said cutting member having a cutting tip at said proximal end having means for mounting an energy source for cutting intraocular tissue and a cutting member collar at a predetermined point between said proximal end and said distal end;
an energy source mounted in said cutting member and adapted to cut intraocular tissue;
a setting dial for moving said input gear to a predetermined distance of rotation;
a lock for maintaining said input gear in said predetermined distance of rotation;
a release for unlocking said lock to permit said spring to return said input gear to its original position along said elliptical path to thereby cause said cutting tip to move along said circular path to perform said continuous curvilinear incision.

13. The device of claim 12, which further includes a housing for enclosing said device; said housing including a rear housing cover for mounting the distal end of said cutting member.

14. The device of claim 12, wherein said corneal, sclearal, or sclero-corneal incision is less than 1.1 mm.

15. The device of claim 14, wherein said cutting member has a diameter of no greater than 1 mm.

16. The device of claim 12, wherein said continuous curvilinear incision may be in the range of about 1.0 nm to about 10.0 mm.

17. The device of claim 12, wherein said energy source is a laser.

18. The device of claim 17, wherein said setting dial includes a stop for limiting the rotation of said cutting means upon release thereon.

19. The device of claim 18, wherein said stop limits said rotation to approximately one revolution.

20. The device of claim 12, wherein said handle is ergonomically designed for the comfortable grasp of the device by the user.

21. The device of claim 12, which further includes a user level for indicating the orientation of the device to the user.
